(19) United States
(12) Patent Application Publication
(10) Pub. No.: US 2006/0058844 A1
(43) Pub. Date: Mar. 16, 2006

(54) Vascular sealing device with locking system

(75) Inventors: John White, Lakeville, MN (US); Andrew Thomas Forsberg, Minneapolis, MN (US)

Correspondence Address: L. Grant Foster;HOLLAND & HART LLP, PO BOX 8749, P.O. Box 8749, Denver, CO, 80201, US

(73) Assignee: St. Jude Medical Puerto Rico B.V.

(21) Appl. No.: 10/939,841

(22) Filed: Sep. 13, 2004

(51) Int. Cl. A61B 17/04

(52) U.S. Cl. 606/232

(57) ABSTRACT

An internal tissue puncture closure method and apparatus provides a locking device for compressing and holding an external component such as a collagen sponge at a puncture situs. The locking device facilitates compression of the external component in a first direction, but prevents or locks against retraction.

### FIELD OF THE INVENTION

The present invention relates to medical devices, and, more particularly, to a vascular puncture closure apparatus.

### BACKGROUND OF THE INVENTION

Various medical procedures, particularly cardiology procedures, involve accessing a corporeal vessel or other lumen through a percutaneous sheath. The access to the vessel necessarily requires the formation of a hole or puncture in the vessel wall so that a medical procedure can be performed. After the particular medical procedure has been performed, the sheath and other tools must eventually be removed from the vessel and the access hole in the vessel wall must be closed.

A number of prior vascular closure devices have been developed to close the hole in the vessel wall. Closing the hole in the vessel wall typically involves packing a resorbable sealing plug at the hole or sandwiching the hole between the sealing plug and an anchor. Examples of prior vascular closure devices are described in U.S. Pat. Nos. 6,179,863; 6,090,130; and 6,045,569, which are hereby incorporated by reference.

However, prior to a successful deployment of the sealing plug or another vascular tool, the introducer must be properly located within the vessel or other lumen. Proper placement of the introducer enables proper placement of the sealing plug or insertion of a vascular tool.

According to conventional techniques, proper placement of the introducer is accomplished with the aid of a puncture locator. Typically, the puncture locator and the introducer are inserted partially through the hole in the vessel wall. The puncture locator includes a fluid communication path between a distal end (where the puncture locator enters the vessel) and a proximal end thereof, such that blood flow can be observed by an operator when the distal end enters the vessel. As the sheath penetrates the vessel wall, blood flows through the fluid communication path and out of a drip hole. Blood continues to flow through the puncture locator until the sheath and/or puncture locator are removed from the vessel. Usually the orientation between the puncture locator and the introducer can be fixed, such that locating the puncture with the puncture locator also properly places the introducer.

To close the puncture following completion of a vascular procedure, the sealing plug is placed at the puncture location via a sealing device that is inserted through the properly placed introducer. The sealing plug is packed at the puncture location by manually tamping the sealing plug toward the hole with a tamping tube. The sealing plug most often provides a sufficient seal of the puncture as a result of the tamping. However, without a retention mechanism, the sealing plug expands, moves, or repositions itself following the tamping operation. Therefore, a suture is usually threaded through the sealing plug and a slipknot is formed proximal to the sealing plug. The slipknot is tightened following the tamping and provides a small surface area that tends to prevent the sealing plug from moving or re-expanding. However, the manufacture of sealing devices with a slipknot is difficult, and a slipknot provides a very small surface area to prevent plug movement. Therefore, the present invention is directed to eliminating, or at least reducing the effects of, one or more of the problems recited above.

### SUMMARY OF THE INVENTION

In one of many possible embodiments, the present invention provides a suture locking system. The suture locking system includes an anchor, a locking apparatus with a first suture passageway, and a suture threaded through the first suture passageway and to the anchor. The first suture passageway of the suture locking system allows relative movement between the suture and the locking apparatus in only one direction. Therefore, the locking apparatus may be advanced toward the anchor along the suture to compress a sealing plug toward an arteriotomy or other internal tissue puncture, but the locking apparatus may not retract.

According to some embodiments, the first suture passageway is substantially linear and tapered from a first end nearest the anchor to a second end farthest from the anchor. The first suture passageway may therefore be conical. The first end of the first suture passageway has diameter larger than an outer diameter of the suture, and the second end of the first suture passageway has a diameter smaller than the outer diameter of the suture. Therefore, the suture is radially compressed as it passes through the first suture passageway in a first direction from the larger to the smaller diameter. The compressing of the suture prevents the suture from retracting through the first suture passageway.

According to other embodiments, the first suture passageway comprises a plurality of flexible, cantilevered levers that are tapered from the first end of the first suture passageway to the second end of the first suture passageway. Accordingly, the cantilevered levers are biased radially inward. According to some embodiments, free ends of the cantilevered levers comprise sharp points that tend to snare the suture if the suture tends to move through the first suture passageway in certain directions.

According to some embodiments the locking apparatus includes a second suture passageway. The suture may pass through the first suture passageway in a first direction, loop through the anchor, and pass through the second suture passageway in a second direction. One or more of the ends of the first and second suture passageways may be beveled to facilitate passage of the suture therethrough.

Another embodiment of the invention provides a tissue puncture closure device for partial insertion into and sealing of an internal tissue wall puncture. The tissue puncture closure device comprises a filament, an anchor for insertion through the tissue wall puncture attached to the filament at a first end of the closure device, a sealing plug disposed proximal of the anchor, and a locking apparatus arranged adjacent to the sealing plug for compressing the sealing plug toward the anchor. The filament is threaded through the locking apparatus and the locking apparatus is movable along the filament toward the sealing plug, but is not movable along the filament away from the sealing plug. A wedge, snare, taper, or one or more cantilevered levers may facilitate the one-way locking movement of the locking apparatus. The tissue puncture closing device may also include a tube slidingly disposed about the filament proximal to the locking apparatus for advancing the locking apparatus along the filament.

Another embodiment provides a tissue puncture sealing device comprising an internal component configured to be positioned against an internal wall of a lumen, and an external component configured to be positioned external to the lumen, such that the external component is operatively connected to the internal component by a suture. The device also includes a locking apparatus positioned adjacent to the external component and disposed about the suture. The locking apparatus is configured to compress and hold the internal and external components together. According to some embodiments the internal component is an anchor and the external component is a collagen sponge. The locking apparatus may comprise a disc with a first suture passageway, the first suture passageway comprising a taper, a step, a snare, or other features that allow the suture to pass therethrough in only one direction.

The invention also provides a method of sealing a puncture in an internal tissue wall accessible through a percutaneous incision. The method includes inserting a closure device at least partially into the percutaneous incision, advancing a one-way hub along a suture, and compressing a sealing plug toward the puncture by the advancing of the one-way hub along the suture.

The foregoing and other features, utilities and advantages of the invention will be apparent from the following more particular description of preferred embodiments of the invention as illustrated in the accompanying drawings.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1A is an assembly view, partly in section, of an internal tissue puncture closure device and an introducer according to one embodiment of the present invention.

FIG. 1B is an enlarged sectional view of the internal tissue puncture closure device of FIG. 1A.

FIG. 2A is a perspective view of a locking apparatus of the internal tissue puncture closure device according to one embodiment of the present invention.

FIG. 2B is an enlarged view, partly in section, taken along the line 2B-2B of FIG. 2A of the locking apparatus shown according to one embodiment of the present invention.

FIG. 3A is a perspective view of another locking apparatus of the tissue puncture closure device without a suture according to one embodiment of the present invention.

FIG. 3B is a sectional side elevation view, taken along the line 3B-3B of FIG. 3A, of the locking apparatus of FIG. 3A without a suture according to one embodiment of the present invention.

FIG. 3C is a side elevation view, partly in section, of the locking apparatus of FIG. 3A with a suture according to one embodiment of the present invention.

FIG. 4A is a perspective view of another locking apparatus of the tissue puncture closure device according to one embodiment of the present invention.

FIG. 4B is a sectional side elevation view, taken along the line 4B-4B, of the locking apparatus according to one embodiment of the present invention.

FIG. 5A is a perspective view of another locking apparatus of the tissue puncture closure device according to one embodiment of the present invention.

FIG. 5B is a sectional side elevation view, taken along the line 5B-5B of FIG. 5A, of the locking apparatus according to one embodiment of the present invention.

FIG. 6 is a side view, partly in section, of the tissue puncture closure device and introducer of FIG. 1A shown in relation to a patient with an anchor deployed according to one embodiment of the present invention.

FIG. 7 is a side view, partly in section, of the tissue puncture closure device and introducer of FIG. 6 shown with the device and introducer being retracted from a percutaneous incision.

FIG. 8 is a side view, partly in section, of the tissue puncture closure device and introducer of FIG. 7 with a tamping tube advancing the locking apparatus.

FIG. 9 is a side elevation view, partly in section, of a sealed tissue puncture following removal of the tissue puncture closure device and introducer.

Throughout the drawings, identical reference numbers designate similar, but not necessarily identical, elements.

### DETAILED DESCRIPTION

The present specification describes techniques and apparatus for closing an internal tissue wall puncture, preferably using a closure device and an introducer, while reducing the effects of sealing plug repositioning. While the methods and devices shown and described below include introducers and puncture sealing devices, the application of a locking apparatus to secure a sealing plug is not limited to these specific devices. The principles described herein may be used to hold a locking apparatus along a suture or other filament for any device, but may be particularly useful to retain a sealing plug at an internal tissue puncture. Therefore, while the description below is directed primarily to arterial procedures, the methods and apparatus may be used according to principles described herein with any filament to limit movement of a locking device to one direction along the filament.

As used in this specification and the appended claims, the term “tissue” means an aggregation of morphologically similar cells and associated intercellular matter acting together to perform one or more specific functions in a body. A “lumen” is any open space or cavity in a bodily organ, especially in a blood vessel. “Linear” means straight, or resembling a line. “Cantilevered” refers to a projecting structure that is supported at one end but not at the other. The words “including” and “having,” as used in the specification, including the claims, have the same meaning as the word “comprising.”

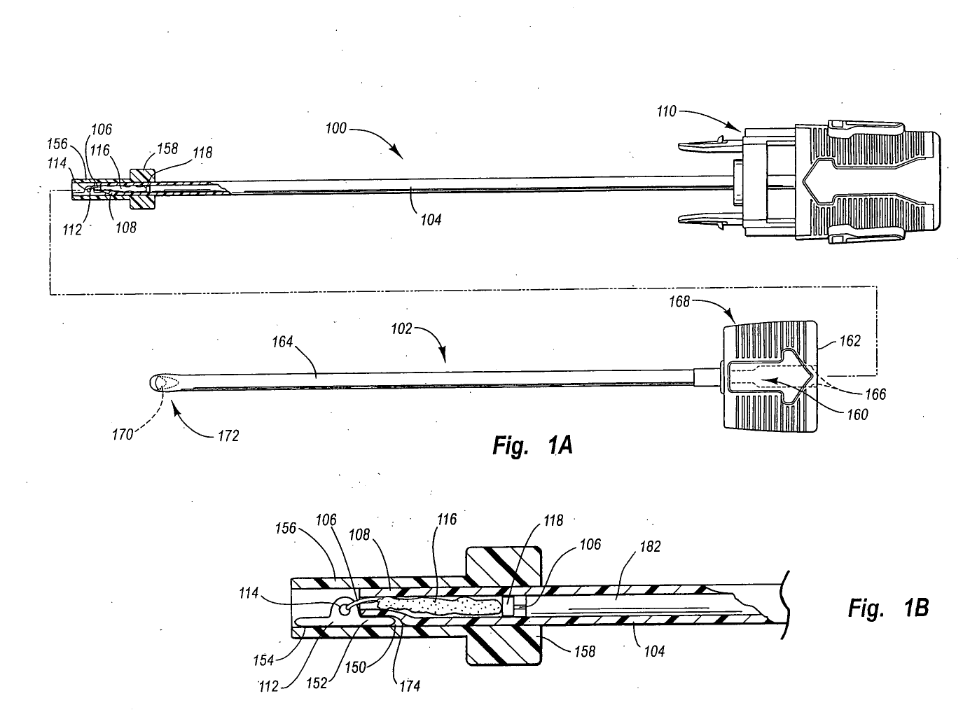

Referring now to the drawings, and in particular to FIGS. 1A-1B, an internal tissue wall puncture closure assembly including a tissue puncture closure device 100 and an introducer 102 is shown according to one embodiment of the present invention. The tissue puncture closure device 100 includes a carrier tube 104 with a filament such as a suture 106 extending at least partially therethrough. According to FIGS. 1A-1B, the suture 106 extends from a first or distal end 108 of the closure device 100 to a second or proximal end 110 of the closure device 100. External to the first or distal end 108 of the carrier tube 104 is an internal component, which according the present embodiment is an anchor 112. The anchor 112 is an elongated, stiff, low-profile member with a protruding eye 114. The anchor 112 is made of a non-hemostatic biologically resorbable polymer.

The suture 106 is also made of a biologically resorbable material and is threaded through the anchor 112. An external component, which, according to the present embodiment, is a biologically resorbable collagen sponge 116 that acts as a sealing plug, is initially disposed within the carrier tube 104 proximal of the anchor 112. The collagen sponge 116 is slidingly arranged about the suture 106 adjacent to a locking apparatus 118 for compressing the collagen sponge 116 toward the anchor 112. According to FIGS. 1A-1B, the collagen sponge 116 is freely arranged about the suture 106.

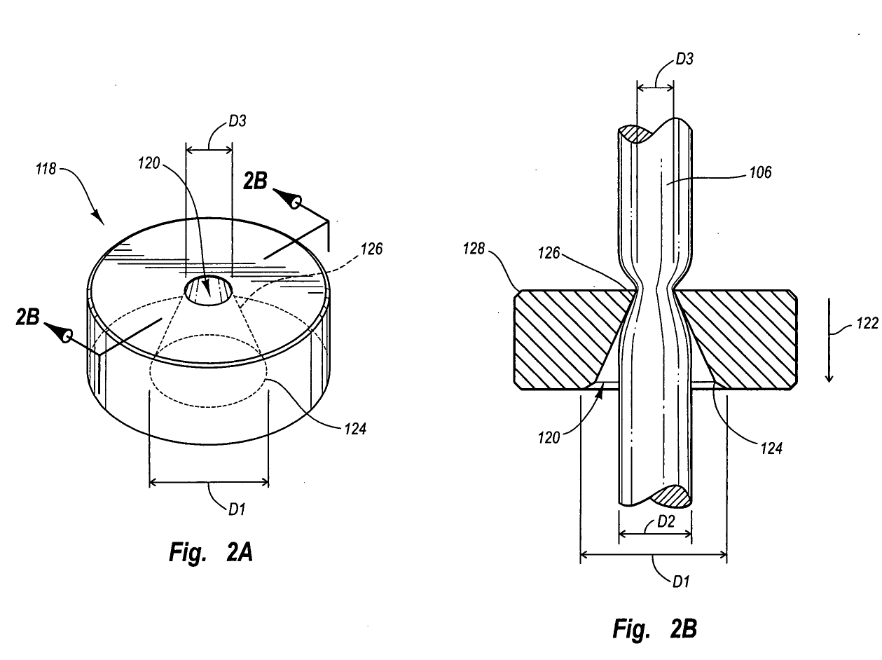

The locking apparatus of FIGS. 1A-1B comprises a hub or a generally cylindrical, one-way locking disc 118. Various embodiments of the disc 118 are shown and described in more detail below with reference to FIGS. 2A-5B. Those of skill in the art having the benefit of this disclosure will recognize that the disc 118 may comprise other non-cylindrical shapes as well, including, but not limited to: polygons and ellipses.

Referring to FIGS. 2A-2B, the suture 106 extends through a first suture passageway 120 disposed in the disc 118. The first suture passageway 120 as shown is substantially linear and concentrically formed in the disc 118. The first suture passageway 120 of FIGS. 2A-2B allows relative movement between the disc 118 and the suture 106 in only one direction. Therefore, according to the embodiment shown, the disc 118 may be advanced in the direction of an arrow 122 to compress the collagen sponge 116 (FIG. 1B), but not in a direction opposite arrow 122. The disc 118 comprises a surface area of greater than approximately 0.001 square inches, which provides significantly more surface holding area than prior knots. For example, according to some embodiments the diameter of the disc 118 is approximately 0.060 inches, and the area is approximately 0.0028 square inches.

The one-way movement between the disc 118 and the suture 106 is facilitated according to FIGS. 2A-2B by sloping, tapering, or stepping the first suture passageway 120. As shown in FIGS. 2A-2B, the first suture passageway 120 is tapered from a first end 124 nearest to the anchor 112 (FIG. 1B) to a second end 126 farthest from the anchor 112 (FIG. 1B). A diameter D1 at the first end 124 of the first suture passageway 120 is larger than a diameter D2 of the suture 106, while a diameter D3 at the second end 126 of the first suture passageway 120 is smaller than the diameter D2 of the suture 106. The diameter D1 at the first end 124 may also be beveled as shown. Accordingly, the disc 118 may easily advance along the suture 106 as the suture 106 enters the first suture passageway 120 at the first end 124, and the taper facilitates passage of the suture 106 through the second end 126. However, as the suture 106 passes through the second end 126, the suture 106 is compressed to the diameter D3. The smaller diameter D3 pinches the suture 106, and because there is no gradual taper in the direction opposite of the arrow 122, the portion of the suture 106 that has already passed through disc 118 is prevented from re-entering the second end 126 without a substantial force. Therefore, the disc 118 may advance easily along the suture 106 in the direction of the arrow 122, but locks itself from opposite movement.

According to some embodiments, the disc 118 is made of a single-piece or unitary construction and preferably made of biologically resorbable polymers. Nevertheless, alternative constructions including multiple components may be used. In addition, according to some embodiments the disc 118 may comprise metals, ceramics, non-biologically resorbable polymers, or combinations thereof.

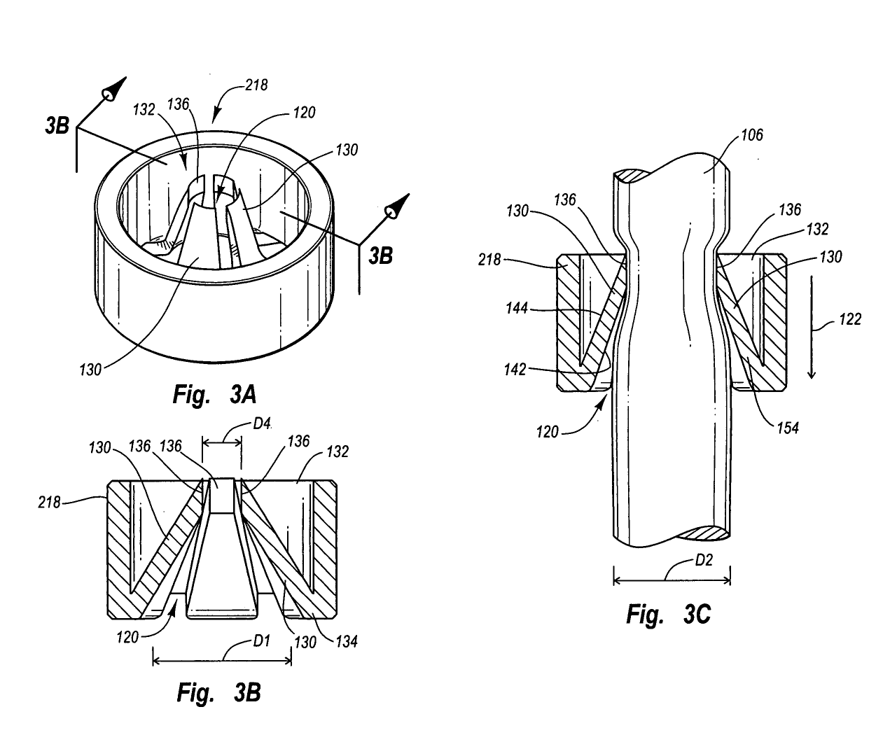

The embodiment of the disc 118 shown in FIGS. 2A-2B is just one of many possible embodiments that may be used to facilitate a one-way suture locking system. Another embodiment of a disc 218 is shown in FIGS. 3A-3C. Referring first to FIG. 3B, the disc 218 is shown in an original position without the suture 106 (FIG. 3C). The disc 218 includes the first suture passageway 120 therethrough, however, the first suture passageway 120 is defined by a plurality of cantilevered prongs or levers 130 which combine to generally form a conical shape. The cantilevered prongs or levers 130 are angled and extend radially inwardly as shown. Each of the cantilevered prongs 130 is disposed in a recess 132 of the disc 218, as more clearly shown in FIG. 3A. First ends 134 of the cantilevered prongs 130 form the diameter D1 of approximately the same dimension as that shown in FIG. 2A. Second ends 136 of the cantilevered prongs 130 form another diameter D4 that is smaller than the diameter D2 of the suture 106 (FIG. 3C). The second ends 136 comprise free ends of the cantilevered prongs 130. The cantilevered prongs 130 form an inner cone that includes both conical inner surfaces 142 and conical outer surfaces 144 recessed within the disc 218. However, the inner and outer conical surfaces 142, 144 may be discontinuous at transitions between the cantilevered prongs 130.

The cantilevered prongs 130 are flexible or resilient such that they expand radially in response to insertion of the suture 106 as shown in FIG. 3C. However, the cantilevered prongs 130 are stiff enough to compress the suture 106 to a diameter of less than the dimension of D2. Therefore, the disc 218 may advance along the suture 106 in the direction of the arrow 122, but as the disc 218 tends to retract in an opposite direction, the cantilevered prongs 130 further compress the suture 106 and lock it from movement in the opposite direction. In addition, the second ends 136 of the cantilevered prongs 130 may be sharp so as to snare the suture 106 and/or further compress the suture 106 at the second ends 136 if the disc 218 tends to move in a direction opposite of the arrow 122 with respect to the suture 106.

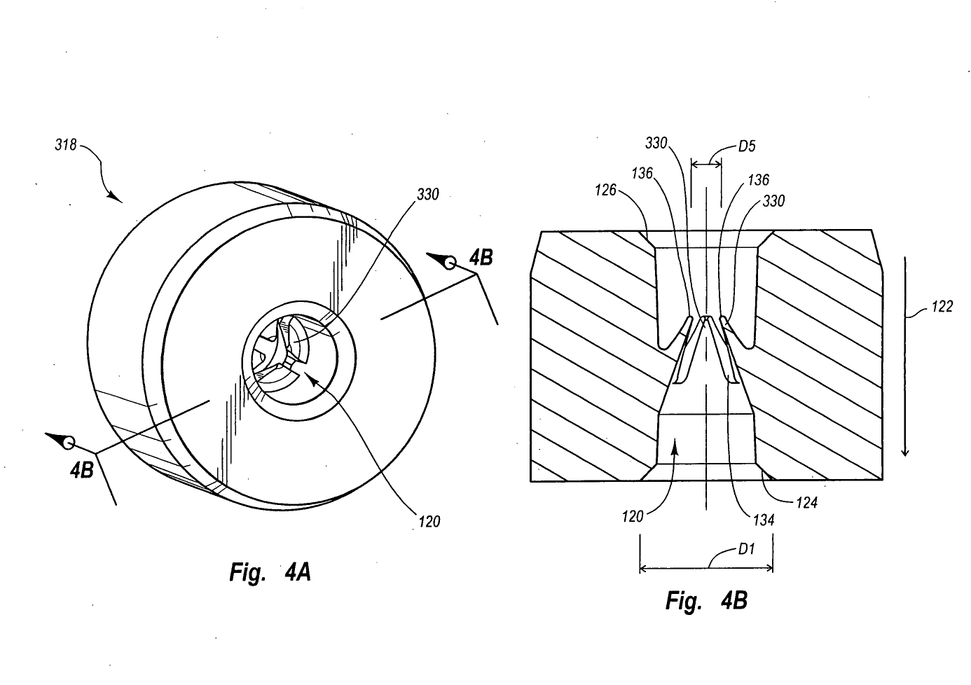

Another embodiment of a disc 318 is shown with reference to FIGS. 4A-4B. The disc of FIGS. 4A-4B also includes the first suture passageway 120, which is shown without the suture 106 (FIG. 1B) for clarity. The disc 318 of FIGS. 4A-4B is similar to the embodiment of FIGS. 3A-3C. However, the embodiment of FIGS. 4A-4C includes cantilevered prongs or snares 330 that are smaller and do not exclusively define the first suture passageway 120. Further, both the first and second ends 124, 126 of the first suture passageway 120 are beveled.

According to the embodiment of FIGS. 4A-4B, the snares 330 are formed approximately mid-way between the first and second ends 124, 126 of the first suture passageway 120 and include sharp points at the second or free ends 136 thereof. The diameter D1 of the first suture passageway 120 is larger than the diameter D2 (FIG. 2B) of the suture at both the first and second ends 124, 126. The free ends 136 of the snares 330, however, form a smaller diameter D5 than that of the suture 106 (FIG. 2B). Both the first and second ends 134, 136 of the snares 330 are contained within the first suture passageway 120, spaced from the first and second ends 124, 126 thereof.

Similar to the cantilevered prongs 130 of FIG. 3A, the prongs or snares 330 of FIGS. 4A-4B are angled radially inwardly to allow the disc 318 to advance along the suture 106 (FIG. 2B) in the direction of the arrow 122. The sharp points at the free ends 136 of the snares 330 tend to grab and lock the disc 318 against motion in a direction opposite of the arrow 122 if the disc 318 should attempt to move relative to the suture 106 (FIG. 2B) in the direction opposite of the arrow 122. The grabbing or locking of the suture 106 (FIG. 2B) by the snares 330 thus provides for only one-way movement between the disc 318 and the suture 106 (FIG. 2B). Accordingly, the disc 318 may advance along the suture 106 (FIG. 1B) toward the anchor 112 (FIG. 1B) to compress the collagen sponge 116 (FIG. 1B), but the disc 318 may not retract along the suture 106 away from the anchor 112 (FIG. 1B).

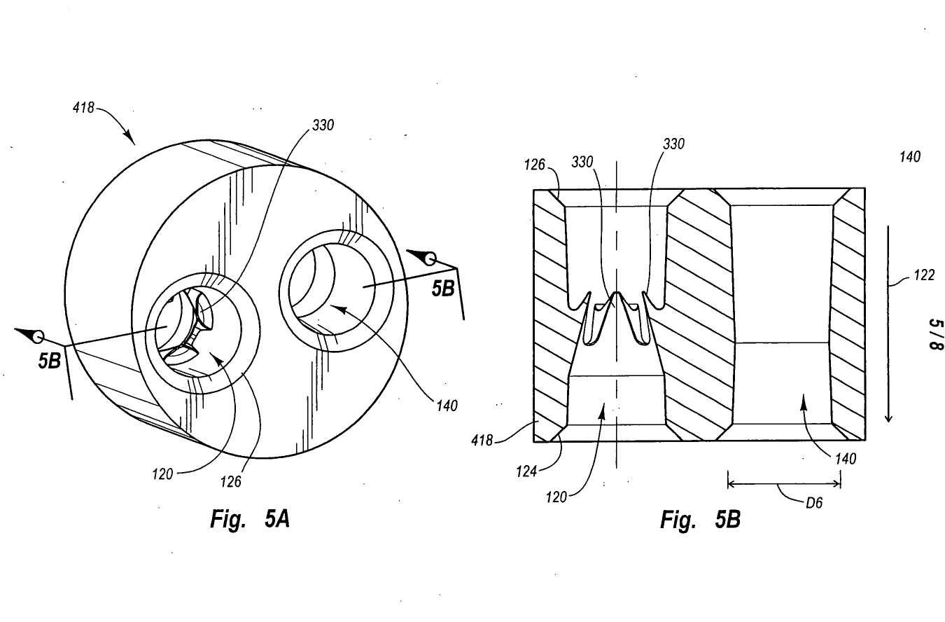

Yet another embodiment of a disc 418 is shown in FIGS. 5A-5B and described in more detail below. The embodiment shown in FIGS. 5A-5B is similar to the embodiment shown in FIGS. 4A-4B. However, a second suture passageway 140 is also included. Further, the first and second suture passageways 120, 140 are arranged off-center. The second suture passageway 140 may comprise a substantially constant diameter D6 as shown. The diameter D6 of the second suture passageway 140 is larger than the diameter D2 (FIG. 2B) of the suture 106 (FIG. 2B) to allow free passage of the suture 106 (FIG. 2B) therethrough in both the direction indicated by the arrow 122 and the direction opposite of the arrow 122.

According to embodiments employing the disc 418 of FIGS. 5A-5B, the suture 106 (FIG. 2B) may pass through the first suture passageway 120 from the first end 124 to the second end 126 in the direction opposite of the arrow 122, loop through the anchor 112 (FIG. 1B), and return through the second suture passageway 140 in the direction of the arrow 122. Accordingly, in operation, the disc 418 may advance along the suture 106 (FIG. 1B) toward the anchor 112 (FIG. 1B) while the length of the suture 106 (FIG. 1B) between the disc 418 and the anchor 112 (FIG. 1B) is reduced by pulling on the suture 106 (FIG. 1B) as it passes through the second suture passageway 140. As with the disc 318 shown in FIGS. 4A-4B, the disc 418 of FIGS. 5A-5B includes the snares 330 to allow only one-way relative motion between the disc 418 and the suture 106 (FIG. 1B).

Any of the discs described above, or any equivalent structure, may be used in operation with the tissue puncture sealing device 100 shown in FIGS. 1A-1B to seal an internal tissue puncture. Referring again to FIGS. 1A-1B, at the distal end 108 of the carrier tube 104 is a nest 150. Prior to deployment of the anchor 112 within an artery or other lumen, the eye 114 of the anchor seats outside the distal end 108 of the carrier tube 104, and one wing 152 of the anchor 112 rests in the nest 150. The nest 150 is generally crushed to a depth such that a surface 154 of the anchor 112 is flush with the outer diameter of the carrier tube 104. The nest 150 is crushed to a length that is preferably longer than the wing 152 of the anchor 112. The anchor 112 may be temporarily held in place in the nest 150 by a bypass tube 156 disposed over the distal end 108 of the carrier tube 104.

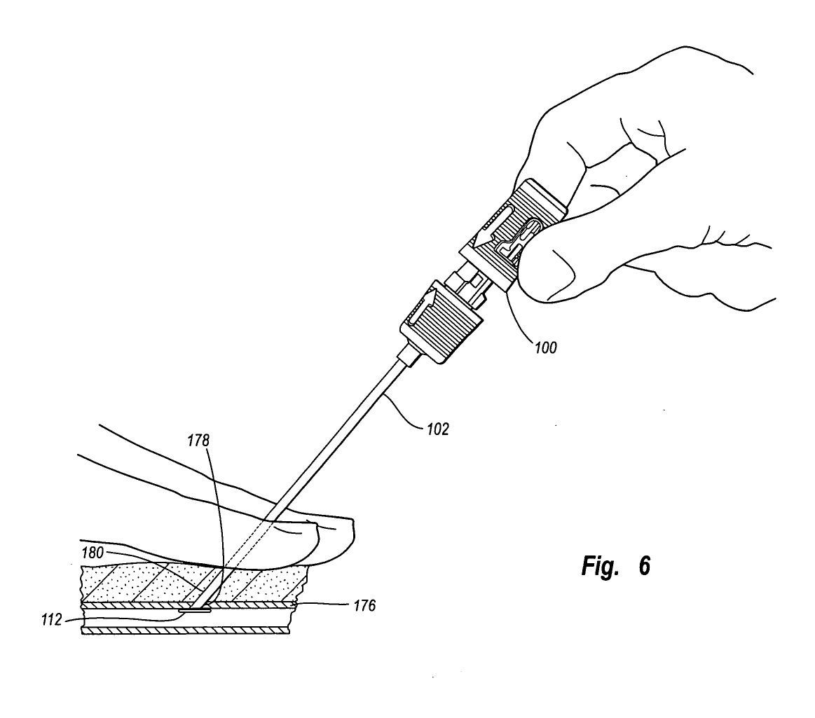

The flush arrangement of the anchor 112 and carrier tube 104 allows the anchor to be inserted into the introducer 102 and eventually through an arterial puncture (shown in FIGS. 6-9). However, the bypass tube 156 includes an oversized head 158 that prevents the bypass tube 156 from passing through an internal passage 160 of the introducer 102. Therefore, as the puncture closure device 100 is inserted into the internal passage 160 of the introducer 102, the oversized head 158 bears against a surface 162 of the introducer 102. Further insertion of the puncture closure device 100 results in sliding movement between the carrier tube 102 and the bypass tube 156, releasing the anchor 112 from the bypass tube 156. However, the anchor 112 initially remains in the nest 150 following release from the bypass tube 156, limited in movement by internal walls of the introducer 102.

The introducer 102 comprises a generally flexible tubular member 164 with a hemostatic valve 166 at a proximal end 168 thereof. The introducer 102 includes a fold 170 disposed at a first or distal end 172 thereof. The fold 170 acts as a one-way valve to the anchor 112. The fold 170 is a plastic deformation in a portion of the introducer 102 that elastically flexes as the anchor 112 is pushed out through the first end 172 of the introducer 102. However, as the anchor 112 passes though and out of the first end 172 of the introducer 102, the fold 170 attempts to spring back to its original deformed position and engages the nest 150. As relative movement between the carrier tube 104 and the introducer 102 continues, the fold 170 traverses a contour 174 of the carrier tube nest 150 in a proximal direction.

Typically, after the anchor 112 passes through the first end 172 of the introducer 102 it enters an artery 176 as shown in FIG. 6 through a puncture 178 via a percutaneous incision 180 in a patient. The puncture 178 in the artery 176 is commonly referred to as an arteriotomy. After the anchor 112 enters the artery 176, the internal tissue puncture closure device 100 is pulled in a proximal direction with respect to the introducer 102. The fold 170 (FIG. 1B) again follows the contour 174 (FIG. 1B) and slides distally between the anchor 112 and the nest 150 (FIG. 1B), causing the anchor to rotate to the position shown in FIG. 6. Accordingly, the anchor 112 is deployed within and aligned with the artery 176.

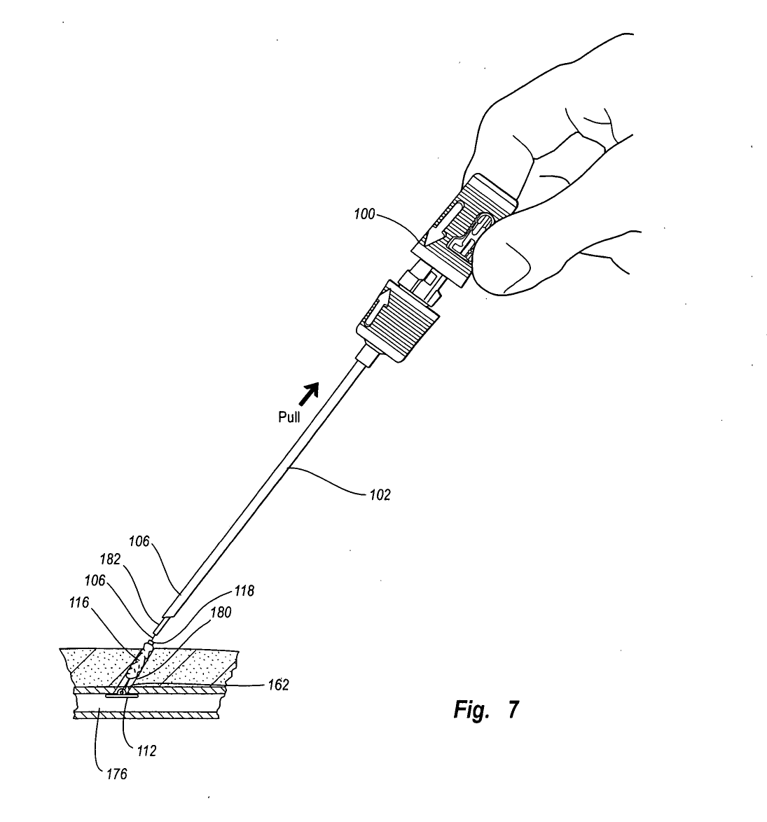

When the anchor 112 is properly positioned inside the artery 176, the closure device 100 and the introducer 102 are withdrawn from the percutaneous incision 180 together as shown in FIG. 7. However, because the anchor 112 is established inside the artery 176, retraction of the introducer 102 and the closure device 100 exposes the collagen sponge 116, the disc 118 (or one of the discs 218, 318, 418), and a tube such a tamping tube 182. Accordingly, the collagen sponge 116 is deposited at an external situs of the puncture 178 in the artery 176 opposite of the anchor 112. The suture 106 is threaded through the tamping tube 182, which is free to move along the suture 106 and is located adjacent to and proximal of the disc 118. The tamping tube 182 may comprise an outer diameter that is larger than an inner diameter of the disc 118 to facilitate advancing the disc 11 8 by pushing the tamping tube 182.

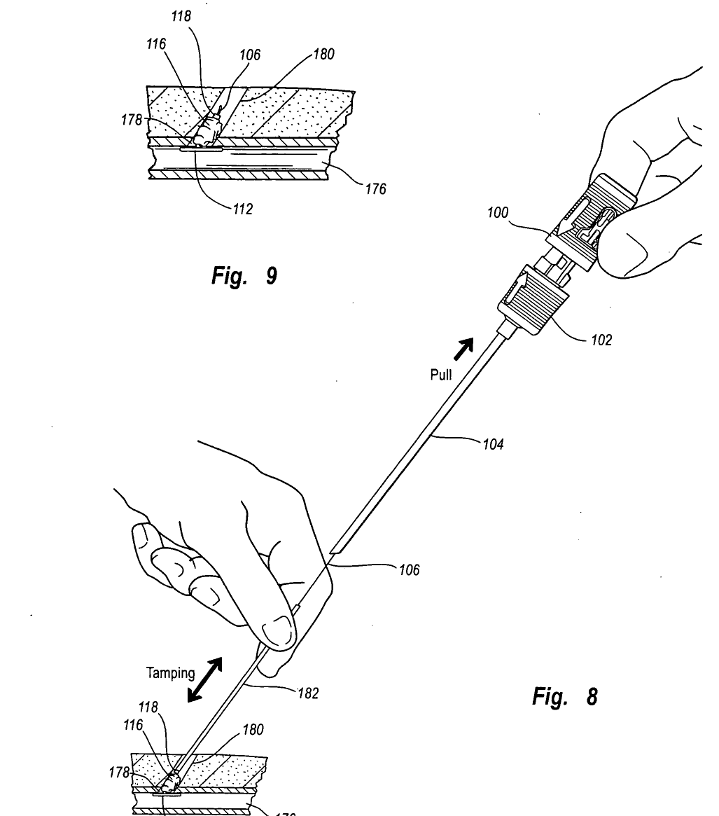

However, depositing the collagen sponge 116 at the puncture 178 does not normally seal the puncture 178. Therefore, according to one embodiment of the present invention, an operator advances the disc 118 along the suture 106 to compress the collagen sponge 116 and sandwich the puncture 178 between the anchor 112 an the collagen sponge 116. According to some embodiments, the disc 118 is advanced by applying a distal force to the tamping tube 182 as shown in FIG. 8. The tamping tube 180 advances the disc 118 distally along the suture 106 in response to the force on the tamping tube 182, and compresses the collagen sponge 116 toward the anchor 112. Tension may be maintained on the suture 106 as the disc 118 is advanced by pulling on the suture 106 or the closure device 100. As discussed above, the geometry of the disc 118 and the suture 106 creates a one-way locking apparatus that allows the disc 118 to advance distally along the suture in a first direction, but prevents the disc 118 from retracting in a second or opposite direction. Therefore, the collagen sponge 116 slides along the suture 106, is compressed toward the anchor 112, and is held in position to seal the puncture 178 by the disc 118.

Following the sealing of the puncture 178 by the collagen sponge 116, the suture 106 may be cut above or proximal to the disc 118. Cutting the suture 106 allows an operator to remove all but the anchor 112, the collagen sponge 116, the disc 118, and a segment of the suture 106 from the patient. Accordingly, as shown in FIG. 9, the puncture 178 is sealed, leaving only the anchor 110, the collagen sponge 114, the disc 116, and the segment of the suture 104 at the puncture 178 site. The components remaining at the puncture 178 site seal the puncture 178 and allow it to heal. Further, each of the anchor 112, the collagen sponge 114, the disc 118, and the segment of suture 106 is preferably biologically resorbable and need not be later removed.

While the invention has been particularly shown and described with reference to embodiments thereof, it will be understood by those skilled in the art that various other changes in the form and details may be made without departing from the scope of the invention.

What is claimed is:

1. A suture locking system, comprising:
an anchor;
a locking apparatus having a first suture passageway therethrough;
a suture threaded through the first suture passageway of the locking apparatus and to the anchor;
wherein the first suture passageway allows the suture to pass therethrough in only one direction.

2. A suture locking system according to claim 1 wherein the first suture passageway comprises a substantially linear passageway tapered from a first end nearest the anchor to a second end farthest from the anchor.

3. A suture locking system according to claim 1 wherein the first suture passageway comprises a conical passageway, wherein a first end of the conical passageway has a larger diameter than an outer diameter of the suture, and a second end of the conical passageway has a smaller diameter than the outer diameter of the suture.

4. A suture locking system according to claim 1 wherein the first suture passageway comprises a plurality of flexible cantilevered levers tapered from a first end of the first suture passageway to a second end of the first suture passageway, and wherein the first end of the first suture passageway comprises a diameter larger than an outer diameter of the suture, and a second end of the first suture passageway comprises a diameter that is smaller than the outer diameter of the suture.

5. A suture locking system according to claim 1 wherein the first suture passageway comprises a plurality of cantilevered prongs tapered from a first end of the first suture passageway to a second end of the first suture passageway, wherein free ends of each of the plurality of cantilevered prongs comprise sharp points.

6. A suture locking system according to claim 1, further comprising a second suture passageway disposed in the locking apparatus, wherein the suture passes through the first suture passageway in a first direction, loops through the anchor, and passes through the second suture passageway in a second direction.

7. A suture locking system according to claim 1 wherein the suture passes through the locking apparatus a single time only.

8. A suture locking system according to claim 1 wherein the locking apparatus comprises a generally cylindrical disc with the first suture passageway centrally disposed therein.

9. A suture locking system according to claim 1 wherein the locking apparatus comprises a unitary construction of biologically resorbable polymer.

10. A suture locking system according to claim 1 wherein the locking apparatus comprises one or more of: metal, ceramic, and a non-biologically resorbable polymer.

11. A suture locking system according to claim 1 wherein the locking apparatus comprises a generally cylindrical outer disc with the first suture passageway centrally disposed therein, wherein the first suture passageway comprises an inner cone, the inner cone comprising both conical inner and outer surfaces, the outer surfaces recessed within the generally cylindrical outer disc.

12. A suture locking system according to claim 1 wherein a first end of the first suture passageway is beveled.

13. A tissue puncture closure device for partial insertion into and sealing of an internal tissue wall puncture, comprising:
a filament;
an anchor for insertion through the tissue wall puncture attached to the filament at a first end of the closure device;
a sealing plug disposed proximal of the anchor, the filament threaded through the anchor;
a locking apparatus arranged adjacent to and proximal of the sealing plug for compressing the sealing plug toward the anchor;
wherein the filament is threaded through the locking apparatus and the locking apparatus is movable along filament toward sealing plug, but is not movable along the filament away from the sealing plug.

14. A tissue puncture closure device for partial insertion into and sealing of an internal tissue wall puncture according to claim 13 wherein the locking apparatus comprises a substantially linear passageway tapered from a first end nearest the anchor to a second end opposite of the anchor, wherein the first end has a diameter larger than an outer diameter of the filament, and a second end has a diameter that is smaller than the outer diameter of the filament.

15. A tissue puncture closure device for partial insertion into and sealing of an internal tissue wall puncture according to claim 13, further comprising first and second filament passageways disposed in the locking apparatus, wherein the filament passes through the first suture passageway in a first direction, loops through the anchor, and passes through the second suture passageway in a second direction.

16. A tissue puncture closure device for partial insertion into and sealing of an internal tissue wall puncture according to claim 13 wherein the locking apparatus comprises a plurality of internal cantilevered prongs tapered from the first end to a second end of the locking apparatus and defining a first filament passageway, wherein free ends of each of the plurality of cantilevered prongs comprise sharp points.

17. A tissue puncture closure device for partial insertion into and sealing of an internal tissue wall puncture according to claim 13 wherein the filament passes through the locking apparatus a single time only.

18. A tissue puncture closure device for partial insertion into and sealing of an internal tissue wall puncture according to claim 13, further comprising a tube slidingly disposed about the filament proximal to the locking apparatus for advancing the locking apparatus along the filament.

19. A tissue puncture closure device for partial insertion into and sealing of an internal tissue wall puncture according to claim 13, further comprising a tube slidingly disposed about the filament proximal to the locking apparatus for advancing the locking apparatus along the filament, wherein the tube comprises an outer diameter that is larger than an inner diameter of the locking apparatus.

20. A tissue puncture sealing device comprising:
an internal component configured to be positioned against an internal wall of a lumen;
an external component configured to be positioned external to the lumen, wherein the external component is operatively connected to the internal component by a suture;
a locking apparatus positioned adjacent to the external component and disposed about the suture;
wherein the locking apparatus is configured to compress and hold the internal and external components together.

21. A tissue puncture sealing device according to claim 20 wherein the tissue puncture is an arteriotomy.

22. A tissue puncture sealing device according to claim 20 wherein the internal component is an anchor and the external component is a collagen sponge.

23. A tissue puncture sealing device according to claim 20 wherein the locking apparatus comprises a disc with a first suture passageway, the first suture passageway comprising a taper or step from a first diameter larger than a diameter of the suture to a second diameter less than the diameter of the suture.

24. A tissue puncture sealing device according to claim 20 wherein the locking apparatus comprises a first suture passageway, the first suture passageway comprising a plurality of prongs arranged substantially in a circle and biased radially inward.

25. A tissue puncture closure device for partial insertion into and sealing of an internal tissue wall puncture, comprising:
a carrier tube having first and second ends;
an anchor disposed outside of the carrier tube at the first end thereof;
a sealing plug disposed inside the carrier tube at the first end thereof;
a one-way sealing plug lock disposed at the first end of the carrier tube for compressing the sealing plug toward the anchor in a first direction and preventing movement of the sealing plug opposite of the first direction.

26. A tissue puncture closure device for partial insertion into and sealing of an internal tissue wall puncture according to claim 25, further comprising a suture attaching the anchor to the one-way sealing plug lock.

27. A tissue puncture closure device for partial insertion into and sealing of an internal tissue wall puncture according to claim 25, further comprising a suture attaching the anchor to the one-way sealing plug lock, and wherein the suture passes through an internal taper of the one-way sealing plug lock, the internal taper comprising a first diameter larger than the suture and a second diameter smaller than the suture.

28. A tissue puncture closure device for partial insertion into and sealing of an internal tissue wall puncture according to claim 25, further comprising a suture attaching the anchor to the one-way sealing plug lock, and wherein the suture passes through an internal passageway of the one-way sealing plug lock, the internal passageway comprising one or more cantilevered prongs biased radially inward.

29. A tissue puncture closure device for partial insertion into and sealing of an internal tissue wall puncture according to claim 25, further comprising a suture attaching the anchor to the one-way sealing plug lock, and wherein the suture passes through an internal passageway of the one-way sealing plug lock, the internal passageway comprising one or more cantilevered prongs biased radially inward and having a sharp free end to allow passage of the one-way sealing plug lock distally but preventing movement of the one-way sealing plug lock proximally by snaring the suture.

30. A tissue puncture closure device for partial insertion into and sealing of an internal tissue wall puncture according to claim 25 wherein the one-way sealing plug lock is disposed within the first end of the carrier tube.

31. A method of sealing an internal tissue puncture, comprising:
providing a closure device having an anchor for insertion through the tissue puncture, a sealing plug disposed proximal of the anchor, and a locking apparatus arranged adjacent to the sealing plug for lockingly compressing the sealing plug toward the anchor;
inserting the closure device partially into the internal tissue puncture;
deploying the anchor;
compressing the sealing plug and the anchor across the internal tissue puncture;
locking the sealing plug and the anchor into a fixed position relative to one another by advancing the locking apparatus along a suture extending between the anchor, the sealing plug, and the locking apparatus.

32. A method of sealing an internal tissue puncture according to claim 31, further comprising inserting the closure device into an introducer.

33. A method of sealing an internal tissue puncture according to claim 31 wherein the locking apparatus comprises a one-way movable disc.

34. A method of sealing an internal tissue puncture according to claim 31 wherein the locking apparatus comprises a one-way disc, and wherein the one-way disc comprises an internal wedge through which the suture can pass in only one direction.

35. A method of sealing a puncture in an internal tissue wall accessible through a percutaneous incision, comprising:
inserting a closure device at least partially into the percutaneous incision;
advancing a one-way hub along a suture;
compressing a sealing plug toward the puncture by the advancement of the one-way hub along the suture.

36. A method of sealing a puncture in an internal tissue wall accessible through a percutaneous incision according to claim 35 wherein the advancing further comprises traversing a plurality of inwardly biased prongs of the one-way hub having sharp free ends along the suture, wherein the inwardly biased prongs are sloped to allow advancement of the hub, but preclude retracting of the hub.

37. A method of sealing a puncture in an internal tissue wall accessible through a percutaneous incision according to claim 35 wherein the inserting further comprises passing the closure device through an introducer and deploying an anchor internal to the puncture.
